(19) United States
(12) Patent Application Publication
(10) Pub. No.: US 2025/0288807 A1
(43) Pub. Date: Sep. 18, 2025

(54) Neural regeneration enhancement device using wireless electrical stimulation based on high-frequency induction coil and conductive hydrogel

(71) Applicants: RESEARCH & BUSINESS FOUNDATION SUNGKYUNKWAN UNIVERSITY, Suwon-si (KR); Korea University Research and Business Foundation, Seoul (KR)

(72) Inventors: MIKYUNG SHIN, Suwon-si (KR); Donghee Son, Suwon-si (KR); Subin Jin, Suwon-si (KR); Sungjun Yoon, Suwon-si (KR); Jong Woong PARK, Seoul (KR)

(21) Appl. No.: 19/075,992

(22) Filed: Mar. 11, 2025

(30) Foreign Application Priority Data
Mar. 13, 2024 (KR) 10-2024-0035468
Feb. 25, 2025 (KR) 10-2025-0024448

(51) Int. Cl. A61N 1/36; A61L 31/04; A61N 1/40

(52) U.S. Cl. CPC A61N 1/36103; A61L 31/041; A61N 1/36171; A61N 1/40

(57) ABSTRACT

The present invention relates to a rehabilitation assistive device utilizing wireless electrical stimulation based on a high-frequency induction coil and conductive hydrogel. Specifically, the present invention concerns a nerve regeneration device using wireless electrical stimulation, which is based on the phenomenon where current is induced within the conductive hydrogel by the electromagnetic waves generated from a coil carrying high-frequency alternating current. The present invention can be applied as a nerve regeneration-promoting system, wherein conductive hydrogel is injected into the damaged area of the subject's nerve tissue using the wireless electrical stimulation device, thereby aiding in nerve regeneration.

### TECHNICAL FIELD

This invention was carried out with the support of the Ministry of Science and ICT of the Republic of Korea under Project No. 00215627. The research management institution for this project is the Pan-Ministerial Regenerative Medicine Technology Development Program. The project title is “Pan-Ministerial Regenerative Medicine Technology Development Program,” and the research task is “Development of an Injectable, Biodegradable, and Neuroadhesive Hydrogel Electrode for Wireless Electrical Stimulation to Promote Minimally Invasive Peripheral Nerve Regeneration.” The host institution is the Sungkyunkwan University Research & Business Foundation, and the research period is from Apr. 1, 2023, to Dec. 31, 2023.

This patent application claims priority to Korean Patent Application No. 10-2024-0035468, filed on Mar. 13, 2024, and Korean Patent Application No. 10-2025-0024448, filed on Feb. 25, 2025 with the Korean Intellectual Property Office, the disclosure of which is incorporated herein by reference.

The present disclosure relates to a rehabilitation assistive device utilizing wireless electrical stimulation based on a high-frequency induction coil and a conductive hydrogel. Specifically, the disclosure pertains to a nerve regeneration device that employs wireless electrical stimulation, wherein an electric current is induced within the conductive hydrogel by electromagnetic waves generated from a coil carrying a high-frequency alternating current.

### BACKGROUND ART

In modern society, cases of nerve damage, such as nerve severance from traffic accidents and industrial injuries, are frequently reported. Such nerve injuries are one of the leading causes of sensory and motor dysfunction and can lead to serious clinical problems, causing significant socio-economic burdens.

Generally, when the damaged area is small (less than 5 mm), spontaneous regeneration is possible. However, when the damaged area is large, regeneration and functional recovery become difficult, necessitating an artificial process for nerve regeneration. Currently known methods for nerve regeneration include autografts, nerve guidance conduits (NGC), stem cells, nerve growth factors, and gene therapy.

Autografting involves harvesting nerves from other areas of the body, such as the sural nerve, which are then directly implanted into both sides of the severed nerve. The success rate of this method is around 50%. However, autografts can cause damage at the nerve harvesting site, and multiple uses of the same nerve are not possible. Additionally, differences in the nerve bundle structures between the donor and regeneration sites, especially the mismatch between motor and sensory nerves, present critical limitations to nerve regeneration and functional recovery at the injury site.

As a result, there has been increasing research and clinical case reports on nerve regeneration guidance conduits as a replacement for autografting. These conduits connect the two sides of the damaged nerve and promote regeneration within the conduit. Currently, commercially available nerve guidance conduits made of single-component materials such as collagen, biodegradable polymers, and silicone-based materials have been developed. However, these conduits still require significant improvement in terms of nerve regeneration speed, functional recovery after regeneration, and effectiveness in long-distance or difficult-to-regenerate nerve repair.

Meanwhile, research has been conducted on the use of conductive materials to promote nerve cell regeneration. In this context, injectable and conductive hydrogels have been studied over the years for their potential to effectively reach inaccessible areas in tissue engineering and soft bioelectronics. However, when using conventional conductive hydrogels, their performance in regenerating damaged nerves is not as effective as autografting, and they fail to address issues related to biostability. Furthermore, conductive hydrogels differ significantly in physical properties from typical biodegradable electrodes, making it challenging to maintain stability when applying long-term currents to stimulate regeneration between damaged nerves.

Additionally, there have been numerous studies and clinical cases reporting the use of electrical stimulation for nerve regeneration to accelerate nerve tissue regeneration and functional recovery. Electrical stimulation of damaged nerves is known to increase neurotrophin expression and promote axonal regeneration, thereby facilitating nerve regeneration. While implantable nerve electrodes and transcutaneous electrical nerve stimulation (TENS) devices are used for nerve electrical stimulation, implantable electrodes require open surgery, and TENS devices need improvements in efficiency for stimulating specific areas.

### PRIOR ART REFERENCES

### Patent Documents

Korean Registered Patent No. 10-2450469 (Registration Date: Sep. 28, 2022)
Korean Published Patent No. 10-2022-0107048 (Publication Date: Mar. 7, 2023)

### DISCLOSURE OF INVENTION

### Technical Problem

The inventors have made diligent research efforts to develop a rehabilitation assistive device for the efficient recovery of motor and sensory functions related to damaged nerves and muscle paralysis. As a result, they discovered that electromagnetic waves generated by a coil carrying a high-frequency alternating current can induce an electric current within a conductive hydrogel, enabling the generation of current wirelessly and at long distances, both inside and outside the body. By demonstrating the applicability of this to a wireless electrical stimulation device, the present disclosure was completed.

Therefore, the objective of the present disclosure is to provide a wireless electrical stimulation device that generates eddy currents within a conductive hydrogel using a coil carrying a high-frequency alternating current.

Another objective of the present disclosure is to provide a nerve regeneration-promoting device using wireless electrical stimulation based on the conductive hydrogel with induced current.

A further objective of the present disclosure is to provide a method for promoting tissue regeneration using the aforementioned wireless electrical stimulation device.

Ultimately, the goal of the present disclosure is to efficiently restore motor and sensory functions related to damaged nerves and muscle paralysis through the nerve regeneration-promoting device utilizing wireless electrical stimulation.

### Solution to Problem

The inventors have made diligent research efforts to develop a rehabilitation assistive device for the efficient recovery of motor and sensory functions related to damaged nerves and muscle paralysis. As a result, they discovered that electromagnetic waves generated by a coil carrying a high-frequency alternating current can induce an electric current within a conductive hydrogel, enabling the generation of current wirelessly and at long distances. They also identified the applicability of this wireless electrical stimulation device for nerve regeneration promotion systems.

According to one aspect of the present disclosure, it provides a wireless electrical stimulation device comprising:
(a) a conductive hydrogel (100) injected into the damaged tissue of the subject;
(b) an induction coil (200) for inducing a current in the conductive hydrogel; and
(c) a high-frequency induction coil controller (300) electrically connected to the induction coil, which controls the size and direction of the current flowing through the induction coil,
wherein the conductive hydrogel (100) and induction coil (200) are spaced apart from each other, and
wherein the high-frequency induction coil controller (300) causes an alternating current to flow through the induction coil (200), thereby inducing a current within the conductive hydrogel (100) due to the electromagnetic waves generated by the coil.

In one embodiment of the present disclosure, the device may be used for electrical stimulation of nerves in a subject with nerve damage, and more specifically, for assisting nerve regeneration in a subject with nerve damage in the lower limbs.

The wireless electrical stimulation device of the present disclosure is characterized by including a conductive hydrogel (100) injected into the damaged tissue of the subject.

The term “conductive hydrogel (100)” used in the present disclosure refers to a material that has high moisture content and adhesive properties, is processed into an ion-conductive substance, and can be attached as an electrode both inside and outside the body. Specifically, it refers to a material that can be used to enhance the electrical signal transmission ability of biological tissue.

The conductive hydrogel is generally a promising material for tissue engineering related to the heart, muscles, nerves, as well as for drug delivery systems responsive to electrical signals and bioelectronics. Various methods have been devised to incorporate conductive additives, such as carbon nanotubes, graphene, noble metal nanoparticles, and conductive polymers, into a crosslinked hydrogel network to manufacture conductive hydrogels. However, these conductive additives have several drawbacks, including poor solubility in hydrophilic environments, cytotoxicity, low dispersion within the hydrogel network, poor gelation, irreversible conductivity, and weak mechanical properties. To overcome these issues, the prior patent of the present disclosure developed a novel electrically conductive hydrogel (IT-IC) using biocompatible natural polymers and provided a method for its production.

In this specification, the term “subject” refers to vertebrates, particularly mammals. The subject includes, for example, livestock, rodents, rabbits, bears, primates, and more specifically, humans, with an even more specific focus on humans with damage to peripheral nerves and/or muscles.

Therefore, in one embodiment of the present disclosure, the wireless electrical stimulation device may apply the novel electrically conductive hydrogel (IT-IC) provided in the prior patent of the present disclosure (e.g., Korean Registered Patent No. 10-2483706, Korean Published Patent No. 10-2025-0000766).

The conductive hydrogel may include hyaluronic acid and noble metal nanoparticles, wherein the hyaluronic acid is linked by biphenyl to form a network, and the noble metal nanoparticles are dispersed on the network.

In a specific embodiment of the present disclosure, the conductive hydrogel (100) is a conductive hydrogel containing hyaluronic acid and noble metal nanoparticles. The hydrogel is prepared by mixing a solvent, hyaluronic acid polymer, and a boronic acid precursor containing a phenyl group to produce a HA-BA polymer solution that includes hyaluronic acid polymer as the main chain and phenylboronic acid as the side chain. Then, a basic solution and noble metal ions are added to the HA-BA polymer solution, resulting in a conductive hydrogel that includes reduced noble metal nanoparticles.

In an embodiment of the present disclosure, the hydrogel features hyaluronic acid polymer as the main chain, with phenylboronic acid containing phenyl groups crosslinked as the side chains.

In another embodiment, the crosslinking degree of the hyaluronic acid polymer and the phenylboronic acid in the HA-BA polymer may range from 2% to 15%, but this is not limiting.

In a further embodiment, the phenylboronic acid may be represented by the following chemical formula.

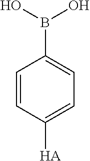

In one embodiment of the present disclosure, the solvent may include water, ethanol, buffer solutions, and combinations thereof, but it is not limited thereto.

Additionally, the hyaluronic acid polymer is a material representative of biocompatible natural polymers, and refers to components that make up the vitreous body, skin, extracellular matrix, etc.

In another embodiment of the present disclosure, in the wireless electrical stimulation device, the hyaluronic acid polymer may be replaced by any material selected from a group consisting of cellulose, collagen, chitin, chitosan, keratin, silk, elastin, and combinations thereof. As long as the material is biocompatible and has conductivity in the form of a polymer, it may be applied in the present disclosure.

Meanwhile, in the preparation of the conductive hydrogel of the present disclosure, the HA-BA polymer solution may additionally include EDC (1-ethyl-3-(3-dimethylaminopropyl)-carbodiimide hydrochloride) and NHS (N-hydroxysuccinimide), but it is not limited thereto.

Specifically, the EDC and NHS refer to substances that remove hydrogen from carboxyl groups, allowing them to bind to amine groups. When EDC removes hydrogen from the carboxyl group of hyaluronic acid, EDC is positioned at the site of the removed hydrogen, thereby binding to hyaluronic acid. Subsequently, the —OH− portion of NHS binds to the site occupied by EDC, and then phenylboronic acid with an amine group binds to hyaluronic acid.

For example, the boronic acid precursor containing a phenyl group may include aminophenylboronic acid (C₆H₄NH₂B(OH)₂), but it is not limited thereto.

The basic solution may include at least one selected from the group consisting of NaOH, KOH, Ba(OH)₂, Ca(OH)₂, NH₄OH, and combinations thereof, but it is not limited thereto. Preferably, the basic solution may include NaOH, but it is not limited thereto.

In the preparation of the conductive hydrogel (100) of the present disclosure, the molar ratio of OH⁻ in the basic solution to the noble metal ions affects the formation of the biphenyl structure. Specifically, when the molar ratio is low, for example, less than 0.1, the deboronation reaction occurs to a lesser extent, resulting in fewer biphenyl structures forming and yielding a hydrogel with a low storage modulus (G′). Conversely, when the molar ratio is excessively high, for example, greater than 10, the reduction of noble metal ions into noble metal nanoparticles by the deboronated polymer is inhibited, leading to noble metal ions being reduced into noble metal nanoparticles due to OH⁻. Specifically, when the noble metal ions are reduced by the deboronated polymer, a biphenyl structure may be formed. However, when the noble metal ions are reduced by OH⁻, the formation of the biphenyl structure by the deboronated polymer may be inhibited.

Thus, the molar ratio of OH⁻ in the basic solution to the noble metal ions may range from about 0.1 to 10, but it is not limited thereto. For example, the molar ratio of OH⁻ in the basic solution to the noble metal ions may be about 0.1 to 10, about 1 to 10, about 2 to 10, about 3 to 10, about 4 to 10, about 5 to 10, about 6 to 10, about 7 to 10, about 8 to 10, about 9 to 10, about 0.1 to 1, about 0.1 to 2, about 0.1 to 3, about 0.1 to 4, about 0.1 to 5, about 0.1 to 6, about 0.1 to 7, about 0.1 to 8, about 0.1 to 9, about 1 to 9, about 2 to 8, about 3 to 7, about 4 to 6, or about 5, but it is not limited thereto.

In this regard, the diameter and size distribution of noble metal nanoparticles formed in the hydrogel of the present disclosure, for example, AuNPs, may vary depending on the molar ratio of the basic solution to the noble metal ions (e.g., the NaOH/Au³⁺ ratio).

In one embodiment of the present disclosure, the noble metal ions may include ions selected from the group consisting of gold (Au), silver (Ag), platinum (Pt), palladium (Pd), iridium (Ir), osmium (Os), and combinations thereof, but it is not limited thereto.

For example, a specific noble metal ion of the present disclosure may be Au³⁺, and the precursor of the Au³⁺ ion may be HAuCl₄, but it is not limited thereto

The conductive hydrogel is characterized by its injectability, allowing for easy application to a desired site. As a result, when injected between the severed ends of a nerve tissue, onto the rough surface of a damaged nerve tissue, or along a three-dimensional (3D) curved structure, the conductive hydrogel exhibits excellent conformal contact, thereby serving as a stable interface material between biological and non-biological substrates.

Additionally, the conductive hydrogel exhibits conductivity as the noble metal nanoparticles, formed by the reduction of noble metal ions during the manufacturing process, provide ionic conductivity. Accordingly, the conductive hydrogel demonstrates biocompatibility, offering the advantage of overcoming the toxicity issues associated with conventional conductive hydrogels.

Moreover, the conductive hydrogel according to the present disclosure includes a biocompatible natural polymer, such as hyaluronic acid, and possesses both biocompatibility and electrical conductivity. This allows it to match the mechanical modulus and electrophysiological characteristics of biological tissues, making it highly useful in tissue engineering and related applications.

Accordingly, in one embodiment of the present disclosure, the conductive hydrogel (100) can be easily injected into the damaged nerve region of a subject and serve as a wireless electrode to facilitate the transmission of electrical stimulation to the nerve tissue. Furthermore, it may contribute to the regeneration of damaged nerve tissue, thereby aiding in the promotion of nerve regeneration.

Meanwhile, in the wireless electrical stimulation device of the present disclosure, the conductivity of the conductive hydrogel (100) increases as the average cross-sectional area of the hydrogel-injected region, perpendicular to the nerve's trajectory, increases. This increase in conductivity can be expressed as a decrease in resistance. For example, hydrogel injected using a thicker needle (18G) exhibits a lower resistance value, and as the amount of injected hydrogel increases, the resistance value decreases.

The resistance of the conductive hydrogel may range from 1 kΩ to 100 kΩ. For instance, it may be about 1 kΩ to 100 kΩ, about 10 kΩ to 100 kΩ, about 20 kΩ to 100 kΩ, about 30 kΩ to 100 kΩ, about 40 kΩ to 100 kΩ, about 50 kΩ to 100 kΩ, about 60 kΩ to 100 kΩ, about 70 kΩ to 100 kΩ, about 80 kΩ to 100 kΩ, about 90 kΩ to 100 kΩ, about 1 kΩ to 10 kΩ, about 1 kΩ to 20 kΩ, about 1 kΩ to 30 kΩ, about 1 kΩ to 40 kΩ, about 1 kΩ to 50 kΩ, about 1 kΩ to 60 kΩ, about 1 kΩ to 70 kΩ, about 1 kΩ to 80 kΩ, about 1 kΩ to 90 kΩ, about 10 kΩ to 90 kΩ, about 20 kΩ to 80 kΩ, about 30 kΩ to 70 kΩ, about 40 kΩ to 60 kΩ, or about 10 kΩ to 50 kΩ. However, it is not limited thereto, and preferably, it may be about 10 kΩ to 50 kΩ.

In the present disclosure, the tangent delta (tan (δ)) of the conductive hydrogel may be greater than 0 and equal to or less than 0.5, but it is not limited thereto.

The term “tangent delta (tan (δ))” refers to the ratio of energy loss due to oscillation and represents the ratio between the storage modulus of a material when it behaves as a pure elastic solid and the loss modulus of a material when it behaves as a pure viscous liquid. A lower tangent delta value indicates a stiffer material. Generally, if the tangent delta of a given gel exceeds 0.5, it is classified as a soft gel, whereas if it is below 0.5, it is categorized as a rigid gel.

In the present disclosure, the elongation at break in the longitudinal direction of the conductive hydrogel may range from 80% to 120%, but it is not limited thereto.

The term “elongation at break” refers to the ratio between the maximum elongation length of the conductive hydrogel before failure and its original length before stretching.

In the present disclosure, when the conductive hydrogel is stretched, its length increases while its cross-sectional area decreases, which may lead to an increase in resistance. In other words, the resistance of the conductive hydrogel may increase according to its elongated length, but it is not limited thereto.

In the wireless electrical stimulation device of the present disclosure, the magnitude of the induced current within the conductive hydrogel is proportional to the amount of hydrogel used. Specifically, when the amount of conductive hydrogel applied in the wireless electrical stimulation device was reduced, a decrease in the magnitude of the induced current within the hydrogel was observed.

The wireless electrical stimulation device of the present disclosure is characterized by including (b) an induction coil (200) that induces a current in the conductive hydrogel (100).

The term “induction coil (200)” as used herein refers to a device that generates eddy currents inside the conductive hydrogel (100) through a magnetic field formed by supplying a specific waveform current.

In one embodiment of the present disclosure, the induction coil (200) is arranged separately from the conductive hydrogel (100).

In this regard, in an exemplary embodiment of the present disclosure, experiments using pig skin tissue confirmed that even when biological tissue is present between the conductive hydrogel and the induction coil, the magnitude of the induced current within the hydrogel remains the same when current flows through the induction coil.

That is, in one embodiment of the present disclosure, the induction coil (200) and the conductive hydrogel (100) are arranged separately from each other, and even if biological tissue exists between them, the magnitude of the induced current in the conductive hydrogel remains the same when current flows through the induction coil.

Accordingly, the conductive hydrogel (100) of the present disclosure can be used in an implanted form within the subject's body, separately from the induction coil (200).

In a specific embodiment of the present disclosure, the conductive hydrogel (100) is injected into a damaged tissue site of the subject, whereas the induction coil (200) is positioned externally to the subject, maintaining a certain physical distance between the hydrogel (100) and the induction coil (200).

In one embodiment of the present disclosure, the induction coil (200) may include a coil fixing member (201) to allow the induction coil to be positioned adjacent to the affected area where the subject's damaged tissue is located. The coil fixing member may be a length-adjustable strap, an elastic band, an adhesive tape, Velcro, a buckle, or a strap provided with an adhesive, but it is not limited thereto.

Meanwhile, in one embodiment of the present disclosure, the induction coil (200) is made of a conductive material selected from metals such as copper, nickel, aluminum, tungsten, stainless steel, iron, silver, gold, lithium, sodium, zinc, cadmium, lead, and their alloys; non-metallic materials containing carbon; and conductive polymer materials.

Specifically, in one embodiment of the present disclosure, copper (Cu) may be used as the material for the induction coil (200), but it is not limited thereto.

The induction coil (200) may be further processed by etching a conductive thin-film coated with a conductive layer, such as a copper foil (or copper-plated) film with a thickness of 0.015 to 0.1 mm.

In one embodiment of the present disclosure, the diameter of the induction coil (200) may range from 0.01 to 0.05 mm, but it is not limited thereto.

In one embodiment of the present disclosure, the induction coil (200) may be formed by winding a metal wire multiple times into a circular shape, but it is not limited thereto.

In one embodiment of the present disclosure, the intensity of the current induced in the conductive hydrogel is proportional to the intensity of the current flowing through the induction coil.

In an exemplary embodiment of the present disclosure, it was confirmed that increasing the current flowing through the induction coil leads to an increase in the current flowing through the hydrogel. Meanwhile, it was also observed that as the current flowing through the induction coil increases, the frequency of the current decreases.

In one embodiment of the present disclosure, the intensity of the current induced in the conductive hydrogel may range from 50 μA to 10 mA, 50 μA to 1 mA, 50 μA to 500 μA, 50 μA to 400 μA, 50 μA to 300 μA, 50 μA to 200 μA, 50 μA to 150 μA, or 50 μA to 100 μA, but it is not limited thereto.

In a specific embodiment of the present disclosure, the current flowing through the induction coil may have an intensity ranging from 100 mA to 3,500 A.

More specifically, the intensity of the current flowing through the induction coil may range from 100 mA to 10 A, 100 mA to 1 A, 100 mA to 800 mA, 100 mA to 700 mA, 100 mA to 600 mA, 100 mA to 500 mA, 100 mA to 400 mA, 100 mA to 300 mA, 100 mA to 200 mA, 1 to 3,500 A, 1 to 3,000 A, 1 to 2,500 A, 1 to 2,000 A, 1 to 1,500 A, 1 to 1,000 A, 1 to 500 A, 1 to 250 A, 1 to 100 A, 1 to 50 A, 50 to 3,500 A, 50 to 3,000 A, 50 to 2,500 A, 50 to 2,000 A, 50 to 1,500 A, 50 to 1,000 A, 50 to 500 A, 50 to 250 A, 50 to 100 A, 100 to 3,500 A, 100 to 3,000 A, 100 to 2,500 A, 100 to 2,000 A, 100 to 1,500 A, 100 to 1,000 A, 100 to 500 A, 100 to 250 A, 250 to 3,500 A, 250 to 3,000 A, 250 to 2,500 A, 250 to 2,000 A, 250 to 1,500 A, 250 to 1,000 A, 250 to 500 A, 500 to 3,500 A, 500 to 3,000 A, 500 to 2,500 A, 500 to 2,000 A, 500 to 1,500 A, 500 to 1,000 A, 1,000 to 3,500 A, 1,000 to 3,000 A, 1,000 to 2,500 A, 1,000 to 2,000 A, 1,000 to 1,500 A, 1,500 to 3,500 A, 1,500 to 3,000 A, 1,500 to 2,500 A, 1,500 to 2,000 A, 2,000 to 3,500 A, 2,000 to 3,000 A, 2,000 to 2,500 A, 2,500 to 3,500 A, 2,500 to 3,000 A, or 3,000 to 3,500 A, but it is not limited thereto.

More specifically, the intensity of the current may range from 100 to 250 A.

Additionally, in a specific embodiment of the present disclosure, the current flowing through the induction coil may have a frequency ranging from 1 Hz to 1,000 KHz.

More specifically, the frequency of the current flowing through the induction coil may range from:
1 Hz to 1,000 KHz, 1 Hz to 500 KHz, 1 Hz to 100 KHz, 1 Hz to 10 KHz, 1 to 1,000 Hz, 1 to 500 Hz, 1 to 250 Hz, 1 to 150 Hz, or 1 to 100 Hz; 100 Hz to 1,000 KHz, 100 Hz to 500 KHz, 100 Hz to 100 KHz, 100 Hz to 10 KHz, 100 to 1,000 Hz, 100 to 500 Hz, 100 to 250 Hz, or 100 to 150 Hz; 150 Hz to 1,000 KHz, 150 Hz to 500 KHz, 150 Hz to 100 KHz, 150 Hz to 10 KHz, 150 to 1,000 Hz, 150 to 500 Hz, or 150 to 250 Hz; 250 Hz to 1,000 KHz, 250 Hz to 500 KHz, 250 Hz to 100 KHz, 250 Hz to 10 KHz, or 250 to 1,000 Hz, 250 to 500 Hz;
500 Hz to 1,000 KHz, 500 Hz to 500 KHz, 500 Hz to 100 KHz, 500 Hz to 10 KHz, or 500 to 1,000 Hz; 1,000 Hz to 1,000 KHz, 1,000 Hz to 500 KHz, 1,000 Hz to 100 KHz, or 1,000 Hz to 10 KHz; 10 KHz to 1,000 KHz, 10 KHz to 500 KHz, or 10 KHz to 100 KHz; 100 KHz to 1,000 KHz, 100 KHz to 500 KHz, or 500 KHz to 1,000 KHz. However, it is not limited thereto, and more specifically, the frequency may range from 10 KHz to 100 KHz.

The wireless electrical stimulation device of the present disclosure is characterized in that the conductive hydrogel and the induction coil are disposed apart from each other.

In another embodiment of the present disclosure, the intensity of the current induced within the conductive hydrogel is inversely proportional to the distance between the induction coil and the conductive hydrogel.

In experiments conducted according to the present disclosure, it was confirmed that as the distance between the induction coil and the conductive hydrogel increases, the intensity of the current flowing through the hydrogel decreases.

In a specific embodiment of the present disclosure, the distance between the induction coil and the conductive hydrogel may range from 1 to 100 mm.

More specifically, the distance may range from:
1 to 100 mm, 1 to 80 mm, 1 to 60 mm, 1 to 40 mm, 1 to 20 mm, or 1 to 10 mm; 10 to 100 mm, 10 to 80 mm, 10 to 60 mm, 10 to 40 mm, or 10 to 20 mm; 20 to 100 mm, 20 to 80 mm, 20 to 60 mm, or 20 to 40 mm; 40 to 100 mm, 40 to 80 mm, or 40 to 60 mm; 60 to 100 mm, 60 to 80 mm; or 80 to 100 mm.

However, it is not limited thereto. As long as the conductive hydrogel and the induction coil are spaced apart within the aforementioned range, an alternating current flowing through the induction coil can generate an electromagnetic wave that induces a current within the conductive hydrogel.

The wireless electrical stimulation device of the present disclosure is characterized by including:
(c) a high-frequency induction coil controller (300) that is electrically connected to the induction coil (200) and adjusts the magnitude and direction of the current flowing through the induction coil.

As described above, the term “high-frequency induction coil controller (300)” in the present disclosure refers to a device that is electrically connected to the induction coil (200) and capable of adjusting the magnitude and direction of the current flowing through it. By controlling the magnitude and direction of the current flowing through the induction coil, the intensity of the current induced in the conductive hydrogel can be adjusted as desired.

In one embodiment of the present disclosure, the wireless electrical stimulation device may further include:
(d) a current-resistance electronic measuring instrument (400) that receives a current signal from the conductive hydrogel (100).

That is, by further including the current-resistance electronic measuring instrument (400), the intensity of the current induced in the conductive hydrogel can be directly measured.

In a nerve regeneration-promoting device including the wireless electrical stimulation device of the present disclosure, the conductive hydrogel (100) plays a key role.

As described above, the wireless electrical stimulation device of the present disclosure includes:
conductive hydrogel (100) injected into the damaged tissue of the subject, an induction coil (200) that induces current in the conductive hydrogel, a high-frequency induction coil controller (300) that directly operates and controls the induction coil.

Specifically, the high-frequency induction coil controller (300) is electrically connected to the induction coil (200) and generates electromagnetic waves by adjusting the magnitude and direction of the current flowing through the induction coil, thus inducing current in the conductive hydrogel (100).

In one embodiment of the present disclosure, the wireless electrical stimulation device induces current wirelessly in the conductive hydrogel connected directly to the damaged nerve tissue of the subject. This current can transmit electrical stimulation to the damaged nerve tissue, aiding the regeneration of the subject's damaged nerve tissue and ultimately promoting nerve regeneration.

In other words, the current induced in the hydrogel by the electromagnetic waves generated by the high-frequency current flowing through the induction coil in the wireless electrical stimulation device can stimulate the damaged nerves.

Thus, when the wireless electrical stimulation device of the present disclosure is applied to a subject who has lost motor function due to damaged peripheral nerves, the conductive hydrogel is injected into the subject's damaged nerve area and serves as a wireless electrode. The device can transmit electrical stimulation induced from the induction coil, which is spaced externally, allowing for the effective operation of a nerve regeneration system.

According to another aspect of the present disclosure, the present invention provides a nerve regeneration-promoting system that includes the wireless electrical stimulation device.

In one embodiment of the present disclosure, the nerve regeneration-promoting system, which includes the wireless electrical stimulation device of the present invention, effectively promotes the regeneration of damaged nerves, thereby restoring not only motor functional abilities but also sensory functional abilities.

In an embodiment of the present invention, the efficacy of the nerve regeneration-promoting system, which includes the wireless electrical stimulation device, was verified through various methods. Specifically, the following evaluations were performed to confirm the nerve regeneration-promoting effect in mice according to the present invention: (i) Sciatic Functional Index (SFI) evaluation, (ii) Tibialis Anterior (TA) muscle weight ratio evaluation, (iii) Compound Muscle Action Potential (CMAP) evaluation in the Tibialis Anterior (TA) muscle, and (iv) sensory responsiveness evaluation.

The Sciatic Functional Index (SFI) refers to an index used to quantitatively evaluate the degree of motor function recovery after sciatic nerve injury in experimental rats or mice. It is typically calculated by measuring the changes in the gait patterns of the experimental animals, such as mice.

In an embodiment of the present invention, it was confirmed that, in mice with sciatic nerve compression injury, the wireless electrical stimulation device of the present invention showed a significantly improved Sciatic Functional Index (SFI) compared to the control group, indicating functional recovery through nerve regeneration promotion.

The measurement of the weight ratio of the Tibialis Anterior (TA) muscle is a key indicator used to quantitatively assess the success of nerve regeneration. Typically, when the nerve is damaged, the TA muscle undergoes denervation atrophy, leading to a decrease in weight. However, when the regenerated nerve reconnects to the muscle, the atrophy begins to recover, and the increase in weight serves as direct evidence of nerve regeneration. In particular, the TA muscle is responsible for dorsiflexion of the foot, and its weight recovery is directly linked to the restoration of motor function.

In an embodiment of the present invention, it was confirmed that, in mice with sciatic nerve compression injury, the wireless electrical stimulation device of the present invention showed a significantly higher weight ratio of the Tibialis Anterior (TA) muscle compared to the control group, indicating the excellent nerve regeneration promotion effect of the present invention.

The Compound Muscle Action Potential (CMAP) refers to an electromyography (EMG) method used to measure the maximum action potential generated in a muscle of interest. It serves as a key indicator for quantitatively evaluating the degree of functional recovery in nerve regeneration, directly reflecting the efficiency of neuromuscular signal transmission. In the present invention, in mice with sciatic nerve compression injury, the wireless electrical stimulation device showed a significantly higher muscle action potential compared to the control group, confirming the positive effect of the present invention on functional recovery through excellent nerve regeneration.

In the nerve regeneration study, the sensory responsiveness measurement in mice is a key indicator used to evaluate the qualitative recovery of nerve fiber regeneration. It provides subtle information on nerve regeneration that cannot be captured by evaluating only motor function recovery. In the present invention, in mice with sciatic nerve compression injury, it was confirmed that the wireless electrical stimulation device showed a higher responsiveness to stimulation compared to the control group, thereby confirming that the nerve regeneration promotion of the present invention also effectively recovers sensory function.

Additionally, in the wireless electrical stimulation device of the present invention, the conductive hydrogel is injected into the damaged nerve area, specifically, into the defect site, where it can also function as a prosthetic. This can prevent the formation of a cavity and the atrophy of surrounding tissues due to tissue defects.

Moreover, the conductive hydrogel can efficiently transmit physiological signals due to its conductivity, thereby aiding in the recovery (rehabilitation) of tissue motor and sensory functions induced by nerve stimulation.

As a result, by utilizing the nerve regeneration promotion system that includes the wireless electrical stimulation device of the present invention, excellent effects can be expected in promoting the regeneration of damaged nerve tissues.

The nerve regeneration promotion system of the present invention incorporates the wireless electrical stimulation device of the present invention, as described above. To avoid excessive complexity in this specification, overlapping content is incorporated by reference, and further detailed descriptions are omitted.

According to another aspect of the present invention, the present invention provides a method for promoting tissue regeneration using the nerve regeneration promotion system described above.

In one embodiment of the present invention, the method includes the following steps:
(a) Injecting conductive hydrogel (100) into the damaged tissue of the subject;
(b) Bringing a wireless electrical stimulation device, which includes an induction coil (200), close to the damaged tissue area where the conductive hydrogel has been injected; and
(c) Applying alternating current (AC) to the induction coil to induce current in the injected conductive hydrogel, thereby stimulating the nerves and muscles.

The invention will be described in detail below for each step.

Step (a): Injecting Conductive Hydrogel (100) into the Damaged Tissue of the Subject

In this step, a biocompatible conductive hydrogel (100) is injected into the tissue where the nerve or muscle has been damaged. The conductive hydrogel of the present invention effectively transmits current to stimulate the damaged nerve.

Additionally, the conductive hydrogel of the present invention is composed of materials that can either be biodegraded in the body or maintain long-term stability. Furthermore, the conductive hydrogel is designed to enhance compatibility with the damaged tissue, ensuring that electrical signals are transmitted uniformly.

In one embodiment of the present invention, methods for injecting the conductive hydrogel include the use of a syringe, catheter, or minimally invasive surgical techniques. Optimal injection conditions can be established by considering the viscosity and coagulation properties of the hydrogel.

Step (b): Approaching the Damaged Tissue Area with the Wireless Electrical Stimulation Device Containing the Induction Coil (200)

In this step, the wireless electrical stimulation device, which includes the induction coil (200), is positioned close to the damaged tissue area.

In one embodiment of the present invention, the induction coil of the wireless electrical stimulation device may be placed on the skin surface above the damaged tissue. For optimal nerve stimulation effects, it is desirable to maintain a certain distance between the conductive hydrogel and the induction coil.

In one embodiment, the distance between the induction coil and the conductive hydrogel may range from about 1 to 100 mm, and more specifically, it may be maintained between about 5 mm and 30 mm, about 5 mm and 20 mm, or about 5 mm and 10 mm. However, these distances are not limiting.

In another embodiment, adjusting the position and angle of the induction coil can maximize the efficiency of the electromagnetic field generated by the coil, thereby allowing for the efficient generation of induced currents within the conductive hydrogel.

In one embodiment, the induction coil may be connected to a skin-attached electrode patch.

In one embodiment of the present invention, the device further includes at least one connector designed to connect to an external device. The connector may include wires that connect to an external power source, delivering current to the induction coil. Additionally, the connector may facilitate data transmission functions by linking the device to an external device.

In another embodiment, the wireless electrical stimulation device may be incorporated into clothing and integrated with conductive fabric. In this case, the wireless electrical stimulation device can be designed in a wearable form, providing continuous electrical stimulation even during the user's activities.

This design offers high convenience in medical and rehabilitation treatments, ensuring that the device can be used effectively while maintaining comfort and ease of use for the wearer.

### Step (c): Applying Alternating Current (AC) to the Induction Coil to Generate Induced Current in the Injected Conductive Hydrogel, Stimulating Nerves and Muscles.

In this step, alternating current (AC) is applied to the induction coil of the wireless electrical stimulation device to create a magnetic field. This magnetic field induces current within the conductive hydrogel, which in turn provides electrical stimulation to the nerve and muscle tissues directly.

In one embodiment of the present invention, the wireless electrical stimulation device includes the induction coil and a high-frequency induction coil controller (300), and the power supplied activates the high-frequency induction coil controller (300) to allow alternating current to flow through the induction coil (200).

In one embodiment of the present invention, the induced current generated in the conductive hydrogel has a range of 50 μA to 10 mA, but this is not limited to these values.

In another embodiment, the frequency of the induced current in the conductive hydrogel is within the range of 1 Hz to 1,000 kHz, but this is not limited to these values.

In one embodiment of the present invention, the wireless electrical stimulation device further includes a current resistance electronic measuring device (400) that can receive the induced current signals generated from the conductive hydrogel.

The stimulation of the nerves and muscles by the induced current in the present invention may trigger the following physiological responses: i) Inducing depolarization of the nerve cell membrane, leading to the generation of action potentials; ii) Activating ion channels within the muscle tissue, causing contraction; iii) Increasing the expression of nerve growth-promoting factors, thereby inducing nerve regeneration.

In this manner, the wireless electrical stimulation device of the present invention can be used in nerve and muscle regeneration therapy, providing effective electrical stimulation without the need for physical connection to external devices.

In one embodiment of the present invention, the stimulation of the nerve and muscle provides at least one of the following: i) Recovery of the function of the damaged nerve; ii) Increased muscle weight at the site of damage; iii) Increased muscle action potential at the damaged site; iv) Increased sensory responsiveness at the damaged site.

In one embodiment of the present invention, the stimulation of the nerves and muscles may be performed for approximately 15 minutes to 2 hours per day, approximately 1 to 3 times per day, approximately 1 to 7 times per week, or over a period of approximately 1 week to 52 weeks or longer. However, it is not limited to these durations.

### Advantageous Effects of Invention

The present invention utilizes electromagnetic waves generated by an alternating high-frequency current flowing through a coil to induce a current within the conductive hydrogel, enabling electrical stimulation of nerve tissues through wireless electrical stimulation. By injecting the conductive hydrogel into the damaged area of the subject's nerve tissue, it can be used as a wireless electrical stimulation device to promote nerve regeneration

### BRIEF DESCRIPTION OF THE DRAWINGS

FIG. 1 is a schematic diagram of the high-frequency induction coil and conductive hydrogel-based wireless electrical stimulation system of the present invention, showing the locations of the current resistance electronic meter, high-frequency induction coil controller, conductive hydrogel, and induction coil.

FIG. 2 is a graph showing the current measured in the conductive hydrogel when an alternating current flows through the high-frequency induction coil.

FIG. 3 is a graph showing the current measured in the conductive hydrogel when the magnitude of the alternating current flowing through the high-frequency induction coil changes. The images on the graph show the alternating current magnitude, frequency, and the light emitted by an LED turned on by the induced current in the conductive hydrogel under each condition.

FIG. 4 is a graph showing the current measured in the conductive hydrogel when the distance between the high-frequency induction coil and the conductive hydrogel changes. The images on the graph show the measurements of the distance between the coil and the hydrogel at the beginning and end of the measurements.

FIG. 5 is a graph showing the difference in the current induced in the conductive hydrogel by the high-frequency induction coil with and without pig skin tissue. The red area indicates the condition where alternating current flows through the high-frequency induction coil.

FIG. 6 is a photo showing the wireless stimulation performed by inducing current in the conductive hydrogel injected into the damage site of a mouse with peripheral nerve compression injury via the high-frequency induction coil.

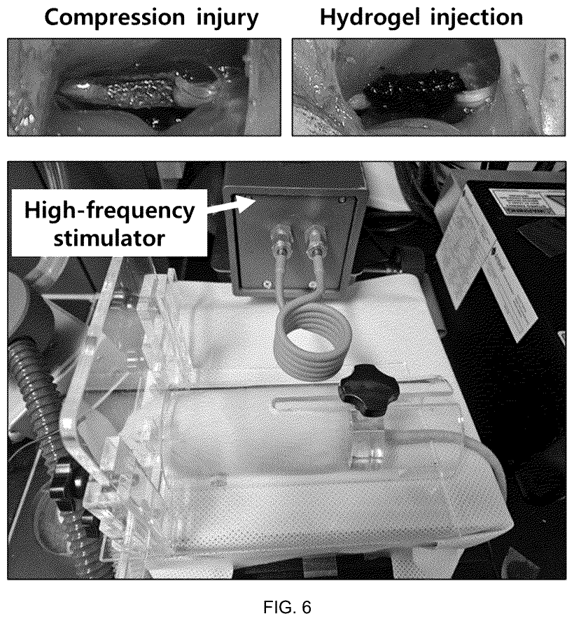

FIG. 7 is a graph showing the Sciatic Functional Index (SFI) assessment of a mouse with a compressed sciatic nerve injury.

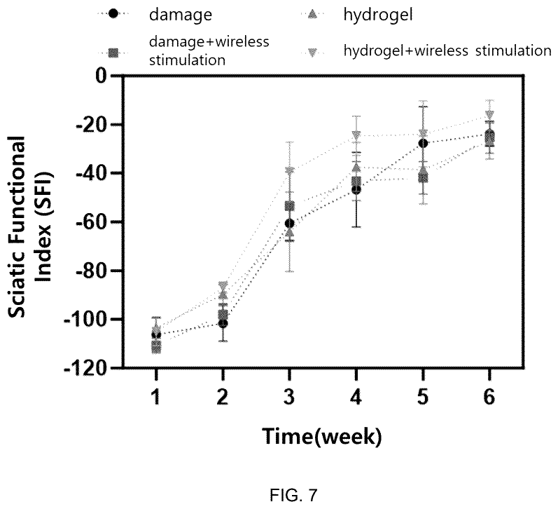

FIG. 8 is a graph evaluating the weight ratio of the tibialis anterior muscle in a mouse with a sciatic nerve compression injury, compared to the opposite leg without nerve damage.

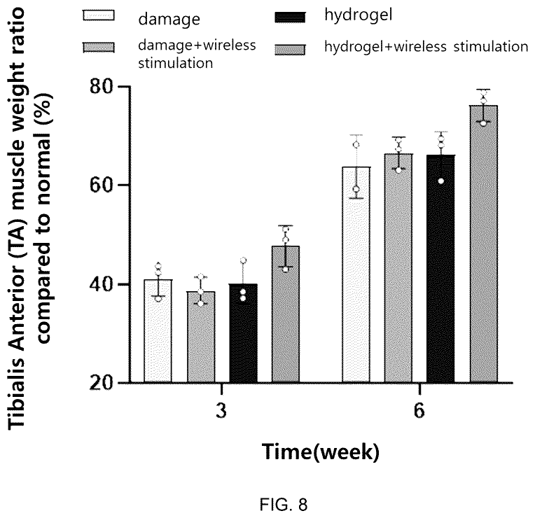

FIG. 9 is a graph showing the Compound Muscle Action Potential (CMAP) evaluation in the tibialis anterior muscle of a mouse with a sciatic nerve compression injury, based on the intensity of the stimulation current.

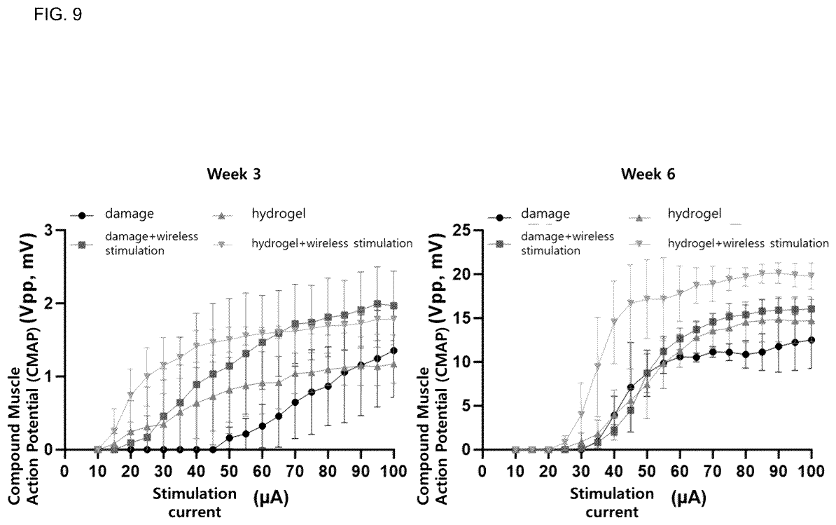

FIG. 10 is a graph evaluating the sensory responsiveness in the damaged plantar region of a mouse with a compressed sciatic nerve injury, based on the number of response areas to physical stimulation.

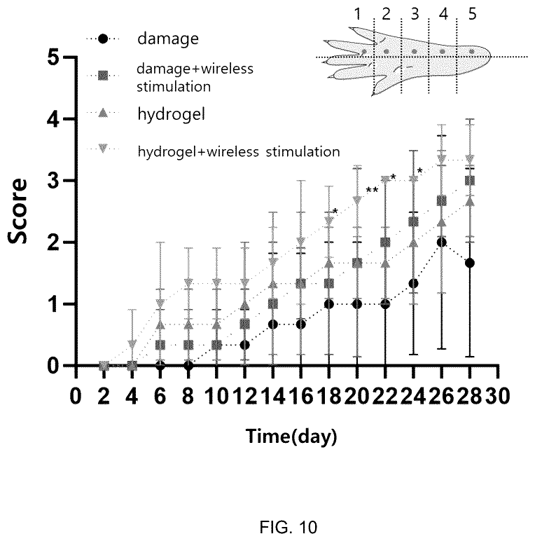

### MODE FOR CARRYING OUT THE INVENTION

The following examples are provided to further explain the present invention in more detail. These examples are solely for the purpose of illustrating the present invention more specifically, and it will be apparent to those skilled in the art that the scope of the present invention is not limited by these examples in accordance with the essence of the invention.

### EXAMPLES

Throughout this specification, the “%” used to indicate the concentration of a specific substance refers to weight/weight % for solid/solid mixtures, weight/volume % for solid/liquid mixtures, and volume/volume % for liquid/liquid mixtures, unless otherwise specified.

### Example 1. Preparation of Conductive Hydrogel (IT-IC)

To prepare a soft conductive hydrogel similar to biological tissue, hyaluronic acid was first dissolved in a solvent, and 4-amino phenyl boronic acid was conjugated to the hyaluronic acid via EDC/NHS coupling reaction to produce the HA-BA polymer. Then, NaOH and HAuCl₄ were added to the HA-BA polymer, and the mixture was stirred to prepare the conductive hydrogel (IT-IC hydrogel) containing gold nanoparticles.

Specifically, 200 kDa hyaluronic acid was dissolved in deionized water (DIW) to a concentration of 10 mg/ml. After adjusting the pH to 5.5, 100 mg of EDC (95.85 mg based on hyaluronic acid) was added to the solution and reacted for 20 minutes. Then, 100 mg of NHS (55.75 mg based on hyaluronic acid) was added, and the reaction continued for 10 minutes. Next, 100 mg of 4-aminophenylboronic acid (86.7 mg based on hyaluronic acid) was added to the solution, which was reacted for 24 hours. Afterward, the solution was placed in a 6 kDa to 8 kDa membrane, and dialysis was performed for 3 days using DIW with a NaCl concentration of 9 g/l. The solution was then frozen at −80° C. for 1 day and freeze-dried at −80° C. for 3 days to obtain the HA-BA polymer, which was dissolved in DIW to a concentration of 20 mg/ml to prepare the HA-BA solution. Subsequently, NaOH solution and HAuCl₄ were added to the HA-BA solution, and the mixture was stirred while maintaining a pH of about 8, resulting in the preparation of the conductive hydrogel (IT-IC hydrogel).

The obtained conductive hydrogel was placed in a plastic tube with a 5 mm diameter and was used in subsequent experiments.

### Example 1. Confirmation of Current Induction Effect by High-Frequency Induction Coil

### Example 1.1. Effect of the Presence or Absence of Current Flowing Through the High-Frequency Induction Coil

A high-frequency alternating current was passed through the induction coil, and the current induction effect in the conductive hydrogel obtained in Example 1 was verified by the electromagnetic waves generated from it.

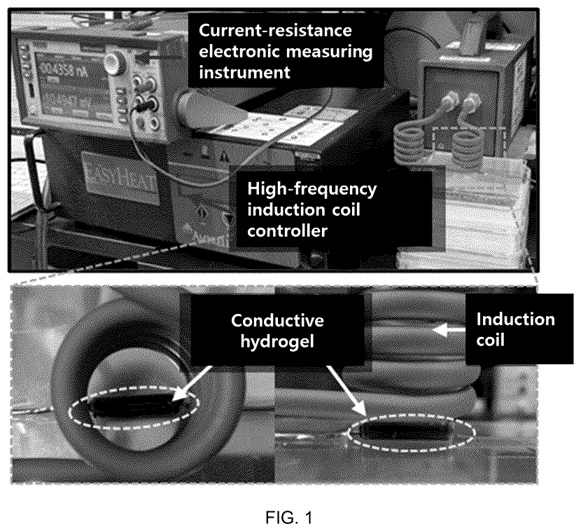

Specifically, as shown in FIG. 1, a plastic tube containing 250 μL of the conductive hydrogel was placed inside the induction coil (arranged with a 1 mm gap). A high-frequency alternating current of 200 A was passed through the induction coil surrounding the conductive hydrogel. The induced current inside the hydrogel was then confirmed using a current resistance electronic measuring instrument.

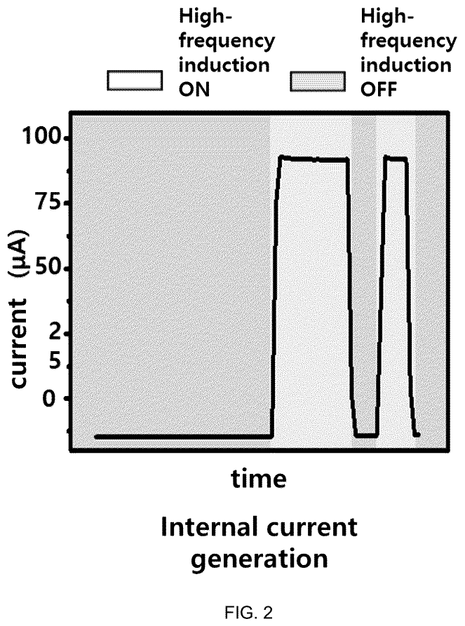

The results are shown in FIG. 2, where it was confirmed that current was induced inside the conductive hydrogel as the high-frequency alternating current flowed through the induction coil. Specifically, when the high-frequency induction current was turned off, no current flowed inside the conductive hydrogel. However, when current flowed through the induction coil, approximately 91 μA of current was measured inside the conductive hydrogel. This allowed for a clear difference in the measured current inside the conductive hydrogel depending on the presence or absence of current flowing through the induction coil.

### Example 1.2. Comparison of the Effect According to the Intensity of Current Flowing Through the High-Frequency Induction Coil

Next, we sought to confirm whether the intensity of the current flowing through the induction coil also affects the intensity of the induced current inside the conductive hydrogel.

To carry out this example, the distance between the induction coil and the hydrogel was set at 10 mm. The intensity of the current induced inside the conductive hydrogel was measured by passing alternating currents of 50, 100, 150, and 200 A through the high-frequency induction coil.

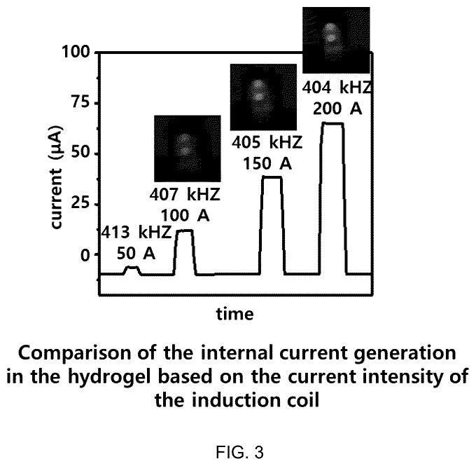

The results are shown in FIG. 3.

As shown in FIG. 3, it was confirmed that as the intensity of the alternating current flowing through the high-frequency induction coil increased, the intensity of the current induced in the conductive hydrogel also increased. Specifically, the induced current increased to approximately 8, 17, 38, and 65 μA, respectively. Additionally, it was observed that as the intensity of the induced current increased, the LED connected to the conductive hydrogel exhibited a clear increase in brightness, with visible differences in the emitted light.

### Example 2. Confirmation of Current Induction Effect Based on the Distance Between the High-Frequency Induction Coil and the Conductive Hydrogel

To confirm the intensity of the induced current inside the hydrogel based on the distance between the high-frequency induction coil and the conductive hydrogel, the distance between the induction coil and the hydrogel was adjusted, and the intensity of the induced current inside the hydrogel was measured.

Specifically, 125 μL of conductive hydrogel was used, and 200 A of alternating current was passed through the induction coil. The distance between the induction coil and the hydrogel was gradually reduced from 50 mm by 2.5 mm increments, i.e., the distance between the conductive hydrogel and the induction coil was decreased, and the intensity of the induced current inside the hydrogel was measured.

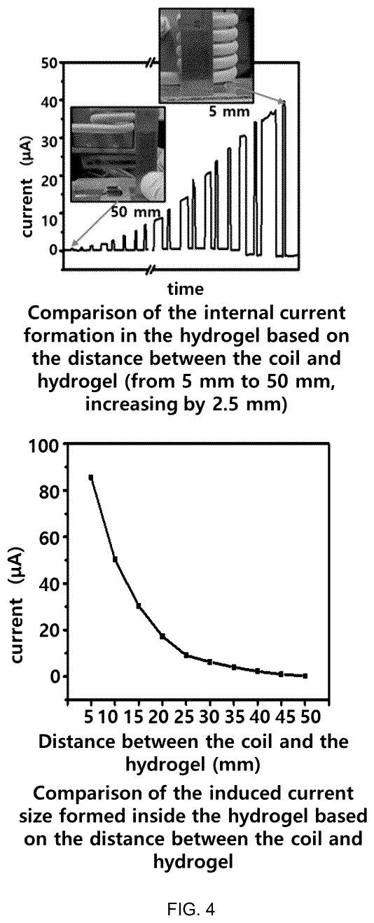

The results are shown in FIG. 4. It was confirmed that as the distance between the induction coil and the conductive hydrogel increased, the intensity of the induced current inside the hydrogel decreased. When the distance between the induction coil and the conductive hydrogel reached 1 mm, about 40 μA of current was induced inside the hydrogel.

This confirmed that alternating current flowing through the high-frequency induction coil generates electromagnetic waves that induce current in the conductive hydrogel placed at a certain distance. Moreover, it was verified that this effect is proportional to the intensity of the current flowing through the induction coil and inversely proportional to the distance between the induction coil and the hydrogel.

### Test Example 1. Confirmation of Current Induction Effect Based on the Presence or Absence of Pig Skin Tissue

To apply the wireless electric induction (stimulation) effect by the high-frequency induction coil of the present invention to biological tissue, the current induction effect inside the hydrogel was confirmed when biological tissue was present between the conductive hydrogel and the induction coil.

Specifically, 375 μL of conductive hydrogel was used, and the distance between the high-frequency induction coil and the hydrogel was set to 6 mm. The conductive hydrogel was covered with pig skin tissue (experimental group: pig skin tissue present/control group: no pig skin tissue). Then, 200 A of alternating current was passed through the induction coil, and the intensity of the current induced inside the hydrogel was measured for each group.

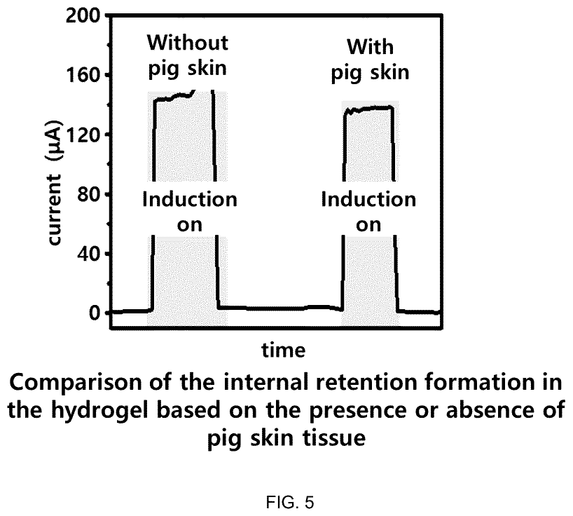

The results are shown in FIG. 5. It was confirmed that regardless of the presence of pig skin tissue, when current flowed through the high-frequency induction coil, current was induced inside the conductive hydrogel. When pig skin tissue was present, about 145 μA was measured, and when there was no pig skin tissue, about 138 μA was measured.

### Test Example 2. Animal Experiment—Verification of Efficacy of Nerve Regeneration Promotion System

The inventors conducted an animal experiment using mice to verify the efficacy of the nerve regeneration promotion system, which includes the wireless electrical stimulation device using the high-frequency induction coil and conductive hydrogel.

As shown in Test Example 6, after inducing compression-type damage to the sciatic nerve of the mice, the hydrogel was injected into the damaged area, and then current was induced in the injected hydrogel through the high-frequency induction coil to perform wireless stimulation. The nerve regeneration efficacy in the mice was evaluated in various ways. The hydrogel was injected with 50 μL, and the high-frequency induction coil was set to flow an alternating current of 290 kHz, 250 A. The distance between the hydrogel and the coil was set to within 5 mm during stimulation. The specific evaluation methods were as follows:
(1) Sciatic Functional Index (SFI) evaluation
(2) Tibialis Anterior (TA) muscle weight ratio evaluation
(3) Compound Muscle Action Potential (CMAP) evaluation in Tibialis Anterior (TA) muscle
(4) Sensory responsiveness evaluation.

All evaluations were performed with four groups, specifically using the mice subjected to wireless stimulation with the high-frequency induction coil and conductive hydrogel after sciatic nerve damage as the experimental group (hydrogel+wireless stimulation). Control groups included the untreated group after damage (damage), the hydrogel-treated group after damage (hydrogel), and the hydrogel group stimulated only with the high-frequency induction coil after damage (damage+wireless stimulation).

### Test Example 2.1. Sciatic Functional Index (SFI) Evaluation

The inventors evaluated the Sciatic Functional Index (SFI) through footprint analysis of mice with compressed sciatic nerve injury.

Specifically, after applying black ink to the soles of the mice's feet, they were allowed to walk straight on a passageway covered with white paper, and their footprints were recorded every week for six weeks. The recorded footprints were scanned, and three measurements were taken using a ruler: PL (distance from the heel to the third toe), TS (distance from the first to the fifth toe), and ITS (distance from the second to the fourth toe). All measurements were performed on both the injured foot (experimental paw, EPL, ETS, and EITS) and the uninjured foot (NPL, NTS, and NITS). The SFI was calculated using the following formula:

$$SFI = - 38.3 \times ( EPL - NPL ) / NPL + 109.5 \times ( ETS - NTS ) / NTS + 13.3 \times ( EITS - NITS ) / NITS - 8.8 .$$

A value of 0 indicates normal function, while a value below −100 indicates complete loss of function in the sciatic nerve.

The results are shown in Test Example 7. As shown in the figure, functional recovery was observed from the mouse footprints by the third week, and notably, the experimental group (the group that underwent both hydrogel and wireless stimulation) exhibited a significantly improved Sciatic Functional Index (SFI) compared to the other control groups, indicating excellent functional recovery. This difference was most prominent in weeks 3 and 4.

### Test Example 2.2. Tibialis Anterior (TA) Muscle Weight Ratio Evaluation

An increase in the weight of the tibialis anterior (TA) muscle is known to serve as direct evidence of nerve regeneration. Therefore, the inventors evaluated the weight ratio of the TA muscle of the injured leg compared to the uninjured contralateral leg at 3 weeks and 6 weeks after inducing sciatic nerve compression injury in mice.

Specifically, after severing the upper and lower ligaments of the TA muscle on the injured side, the muscle was detached from the tibia, and the fascia was removed before weighing. Similarly, the TA muscle of the uninjured normal leg was also weighed. The muscle weight ratio of the tibialis anterior was calculated using the following formula. A weight ratio closer to 100% indicates that the recovery from nerve damage is approaching normal levels.

$$TA Muscle Weight Ratio ( \% ) = ( Weight of muscle from the injured nerve / Weight of muscle from the uninjured nerve ) \times 100$$

The results are shown in Test Example 8. As shown in the figure, compared to the other control groups, the experimental group (the group receiving both hydrogel and wireless stimulation) exhibited a higher TA muscle weight ratio.

### Test Example 2.3. Evaluation of Compound Muscle Action Potential (CMAP) in Tibialis Anterior Muscle

The inventors aimed to quantitatively evaluate the functional recovery of nerve regeneration by performing electrical stimulation on the nerves of mice after sciatic nerve compression injury. The compound muscle action potential (CMAP) in the tibialis anterior (TA) muscle was assessed according to the intensity of the stimulation current at 3 weeks and 6 weeks post-injury.

Specifically, in the peripheral nerve injury model, a soft cuff-type electrode was placed at the proximal site of the injury to apply electrical stimulation, and a needle-type electrode was inserted into the anterior tibialis muscle, which is connected to the stimulated nerve, to measure the compound muscle action potential in response to the electrical stimulation. Measurements were taken at week 0 (before peripheral nerve injury) and again at 3 weeks and 6 weeks post-injury to compare the results and evaluate the recovery of motor nerve function.

The groups measured were: peripheral nerve injury group, peripheral nerve injury with conductive hydrogel group, peripheral nerve injury with RF coil stimulation without hydrogel, and peripheral nerve injury with both conductive hydrogel and RF coil electrical stimulation. These groups were divided into two subgroups for measurements at 3 weeks and 6 weeks, and the results were compared.

The results are shown in Test Example 9. As shown in the figure, compared to the other control groups, the experimental group (the group receiving both hydrogel and wireless stimulation) exhibited higher muscle action potentials and reached the maximum action potential at a lower stimulation current.

### Test Example 2.4. Sensory Responsiveness Evaluation

The inventors aimed to evaluate the qualitative recovery of nerve fiber regeneration by performing physical stimulation on the damaged area of the mouse's paw at 2-day intervals after sciatic nerve compression injury. Sensory responsiveness was then assessed based on the number of responsive areas.

Specifically, the mouse's paw was divided into five vertical sections, and the response of each section was scored as 0 or 1. The mouse was placed on a grid-shaped metal mesh, and a 31G needle attached to a 1 mL syringe was bent at a 90-degree angle. The mouse's paw sections were lightly pricked, and the response was observed. The total response score was calculated by summing the scores of the individual sections.

The results are shown in Test Example 10. As shown in the figure, compared to the other control groups, the experimental group (the group receiving both hydrogel and wireless stimulation) demonstrated higher responsiveness to the stimulus.

Therefore, considering the results as a whole, the conductive hydrogel applied to biological tissue and the wireless electrical induction (stimulation) via the high-frequency induction coil significantly promoted the recovery and regeneration of damaged nerves. This suggests that the wireless electrical stimulation system of the present invention can be effectively used in the treatment of damaged nerve areas.

### DESCRIPTION OF SYMBOLS

1: Wireless electrical stimulation device
100: Conductive hydrogel
200: Induction coil
300: High-frequency induction coil controller
400: Current resistance electronic meter

1. A wireless electrical stimulation device comprising:
(a) conductive hydrogel (100) injected into the damaged tissue of a subject;
(b) an induction coil (200) that induces current in the conductive hydrogel; and
(c) a high-frequency induction coil controller (300) electrically connected to the induction coil and controlling the magnitude and direction of the current flowing through the induction coil,
wherein the conductive hydrogel (100) and induction coil (200) are arranged in spaced apart, and an alternating current is passed through the induction coil (200) by the high-frequency induction coil controller (300), whereby current is induced within the conductive hydrogel (100) by the electromagnetic waves generated therefrom.

2. The wireless electrical stimulation device of claim 1, further comprising:
(d) a current resistance electronic meter (400) for receiving current signals from the conductive hydrogel.

3. The wireless electrical stimulation device of claim 1, wherein the current flowing through the induction coil has a magnitude ranging from 1 to 3,500 A.

4. The wireless electrical stimulation device of claim 1, wherein the current flowing through the induction coil has a frequency ranging from 1 Hz to 1,000 KHz.

5. The wireless electrical stimulation device of claim 1, wherein the distance between the induction coil and the conductive hydrogel ranges from 1 to 100 mm.

6. The wireless electrical stimulation device of claim 1, wherein the conductive hydrogel is a biocompatible natural polymer containing noble metal nanoparticles,
wherein the biocompatible natural polymer is connected by biphenyl to form a network, and the noble metal nanoparticles are dispersed on the network.

7. The wireless electrical stimulation device of claim 6, wherein the natural polymer is selected from the group consisting of cellulose, collagen, chitin, chitosan, keratin, silk, elastin, hyaluronic acid, and combinations thereof.

8. The wireless electrical stimulation device of claim 6, wherein the noble metal nanoparticles are selected from the group consisting of silver (Ag), gold (Au), platinum (Pt), palladium (Pd), iridium (Ir), osmium (Os), and combinations thereof.

9. A nerve regeneration promotion device comprising the wireless electrical stimulation device of claim 1.

10. A method for promoting the regeneration of damaged tissue, comprising the following steps:
(a) injecting a conductive hydrogel (100) into the damaged tissue of a subject;
(b) positioning a wireless electrical stimulation device, comprising an induction coil (200), adjacent to the tissue region into which the conductive hydrogel has been injected; and
(c) applying an alternating current to the induction coil to induce current within the injected conductive hydrogel, thereby stimulating the nerves and muscles.

11. The method of claim 10, wherein the wireless electrical stimulation device comprises an induction coil and a high-frequency induction coil controller (300), and the applied power operates the high-frequency induction coil controller (300) to cause alternating current to flow through the induction coil (200).

12. The method of claim 10, wherein the wireless electrical stimulation device further includes a current resistance electronic meter (400), and receives the induced current signals generated from the conductive hydrogel.

13. The method of claim 10, wherein the induction coil of the wireless electrical stimulation device can be positioned on the surface of the skin over the damaged tissue.

14. The method of claim 10, wherein the device further includes at least one connector designed to connect with an external device.

15. The method of claim 13, wherein the connector is connected to an external power source and includes a wire for transmitting current from the external power source to the induction coil.

16. The method of claim 10, wherein the wireless electrical stimulation device is introduced into clothing and integrated with conductive fabric, in a wearable form.

17. The method of claim 10, wherein the induced current generated in the conductive hydrogel has an intensity of 50 μA to 10 mA.

18. The method of claim 10, wherein the frequency of the induced current generated in the conductive hydrogel is 1 Hz to 1,000 KHz.

19. The method of claim 10, wherein the stimulation of the nerves and muscles provides at least one of:
i) functional recovery of the damaged nerve,
ii) increased weight of the damaged muscle,
iii) increased muscle action potential of the damaged muscle, and
iv) increased sensory responsiveness of the damaged area.

20. The method of claim 10, wherein the stimulation of the nerves and muscles is performed for about 15 minutes to about 2 hours per day, about 1 to 3 times per day, about 1 to 7 times per week, or for a period of about 1 week to about 52 weeks or longer.
